(19) United States
(12) Patent Application Publication
(10) Pub. No.: US 2009/0105740 A1
(43) Pub. Date: Apr. 23, 2009

(54) Facial Bone Surgery Apparatus For Medical

(75) Inventors: HeeYoung Lee, Kunsan (KR); JunSeok Lee, Busan (KR)

Correspondence Address: SUMMA, ADDITON & ASHE, P.A., 11610 NORTH COMMUNITY HOUSE ROAD, SUITE 200, CHARLOTTE, NC, 28277, US

(73) Assignee: Medikan Co., Ltd, Busan (KR)

(21) Appl. No.: 12/349,814

(22) Filed: Jan. 7, 2009

(30) Foreign Application Priority Data
Jul. 10, 2006 (KR) KR 1020060064612
Jul. 10, 2007 (KR) PCT/KR2007/003341

(51) Int. Cl. A61B 17/14; A61B 17/00; A61B 17/32

(52) U.S. Cl. 606/177; 606/79; 606/82; 606/167

(57) ABSTRACT

The present invention relates to a facial bone surgery apparatus for medical treatment assembled to a hand piece having a straight reciprocating part, a saline solution supply part and a foreign substance discharge part, which includes: an inner pipe reciprocally movable together the straight reciprocating means and having a hollow shape of a soft flexible material; an outer pipe connected to a main body of the hand piece and installed at an outer periphery of the inner pipe to guide reciprocal movement of the inner pipe, and having a bent section formed between both ends thereof, the outer pipe being formed of a hard material; and a cutting tool assembled at an end of the inner pipe to reciprocate therewith and reciprocally guided by the outer pipe.

### CROSS-REFERENCE TO PRIORITY APPLICATIONS

This application is a continuation of and claims priority to the commonly assigned Korean Application 10-2006-0064612 (filed Jul. 10, 2006, in the Korean Patent Office) and the commonly assigned International Patent Application No. PCT/KR2007/003341 (filed Jul. 10, 2007, in the Korean Patent Office), both of which are hereby incorporated by reference in their entirety.

### FIELD OF THE INVENTION

The present invention relates to a facial bone surgery apparatus for medical treatment, and more particularly, to a facial bone surgery apparatus for medical treatment coupled to a medical hand piece including a straight reciprocating means, a saline solution supply means and a foreign substance discharge means, and used for contour correction such as a reduction of a lower jawbone or cheekbone and removal of a bone tumor or bone spur.

### BACKGROUND

A surgery apparatus for performing bone amputation or bone cutting can generally be classified into an oscillating sagittal saw method and a reciprocating saw method. Since the oscillating sagittal saw method is designed such that a saw blade moves with a certain angle, extension or reduction in length of the saw blade may cause variation of operation sections and cutting power, thereby making it difficult to cut a portion spaced apart from an incision part.

In addition, in contrast to the oscillating sagittal saw method, since the reciprocating saw method is designed such that a saw blade reciprocally moves within a certain range regardless of the length of the saw blade, the cutting power is uniformly maintained to secure safety and accuracy during surgery, and thus, the reciprocating saw method is widely used nowadays.

As an example of the surgery apparatus using the reciprocating saw method, Korean Patent Registration No. 505133 has been proposed by the inventor. The above-mentioned Korean Patent discloses a facial bone surgery apparatus coupled to a medical power hand piece including a straight reciprocating means, a saline solution supply means and a foreign substance removal means.

The facial bone surgery apparatus includes a long, thin and straight rod. One end of the rod is connected to a reciprocating shaft of a reciprocating means installed at a hand piece to reciprocate. The other end of the rod has a cutting blade, and the cutting blade has holes formed between teeth thereof. The holes are in communication with a foreign substance suction path coupled to an external suction unit to discharge foreign substances such as bone fragments sucked through the holes between the cutting teeth to the exterior.

Further, the cutting blade of the facial bone surgery apparatus includes a saline solution supply unit connected to an external saline solution supply unit. After the surgery apparatus is inserted through a small incision in the skin near the lower jawbone, a saline solution is supplied to the incision through the saline solution supply path to perform the bone cutting. Then, the cut bone fragments are discharged with the saline solution to the exterior through the foreign substance suction path connected to the suction unit, thereby performing a continuous bone cutting operation.

However, because one end of the long, thin and straight rod is integrally formed with the cutting blade, the cutting blade is moved in the same or a similar direction in which the hand piece approaches the cut part, i.e., along a reciprocating axis of the hand piece. Therefore, in the case that various approach angles and various cutting angles are required during the approach and entry of portions of the apparatus through the limited incision or if a surgeon is faced with a limited approach angle, such as during facial bone surgery, it was difficult to perform the surgical operation.

### SUMMARY OF THE INVENTION

In order to solve the problems, it is an object of the present invention to provide a facial bone surgery apparatus for medical treatment having a bent section formed at an insertion part to be inserted into an incision part to enable power transmission through the bent section, such that a cutting operation can be performed in a different direction than a reciprocating direction of a straight reciprocating member installed at a hand piece, thereby enabling surgical operations under the condition of a limited incision or approach angle.

The foregoing and/or other objects of the present invention may be achieved by providing a facial bone surgery apparatus for medical treatment assembled to a hand piece having a straight reciprocating means, a saline solution supply means and a foreign substance discharge means, which comprises: an inner pipe reciprocally movable together with the straight reciprocating means and having a hollow shape of a soft flexible material; an outer pipe connected to a main body of the hand piece and installed at an outer periphery of the inner pipe to guide reciprocal movement of the inner pipe, and having a bent section formed between both ends thereof, the outer pipe being formed of a hard material; and a cutting tool assembled at an end of the inner pipe to reciprocate therewith and reciprocally guided by the outer pipe.

In addition, the facial bone surgery apparatus may further include a straight guide member fixed to an outer surface of the cutting tool to be press-fitted into the outer pipe, wherein a cutting blade direction setting structure is formed between the straight guide member and the outer pipe to vary the direction of a cutting blade of the cutting tool in a circumferential direction about an assembly axis of the cutting tool.

Further, the cutting blade direction setting structure and the straight guide member may have a polygonal outer cross-section.

Furthermore, reinforcement bushings may be formed at outer peripheries of both ends of the inner pipe.

In addition, a saline solution supply path may be formed in a gap between the outer pipe and the inner pipe, and may be in communication with a gap formed between the outer pipe and the straight guide member to supply a saline solution.

Further, a foreign substance suction path may be formed in the inner pipe, and a through-hole may be formed at the cutting tool to be in communication with the foreign substance suction path of the inner pipe.

Furthermore, a connection structure of the outer pipe and the hand piece may include: a fixing support fixed to an outer periphery of the outer pipe; a connection tube detachably coupled to the main body of the hand piece at one side and having a threaded part formed at the other side; a fixing nut hooked by the fixing support and threadedly engaged with the threaded part of the connection tube to couple the outer pipe to the connection tube; and a packing for maintaining a watertight seal between the fixing nut, the outer pipe and the connection tube.

In addition, a bending direction setting structure may be formed between the fixing support and the connection tube to vary an assembly angle of the outer pipe in a circumferential direction about an assembly axis of the outer pipe.

Further, the bending direction setting structure may include: a plurality of insertion grooves formed at the fixing support of the outer pipe in a circumferential direction about an assembly axis thereof; and an insertion projection formed at the connection tube to be press-fitted into the insertion grooves.

Furthermore, a connection structure of the inner pipe and the hand piece may include: an adaptor for connecting the straight reciprocating member of the straight reciprocating means installed at the hand piece to the inner pipe and communicating the foreign substance suction path of the hand piece with the foreign substance suction path of the inner pipe; and a locking nut for threadedly engaging the adaptor with the straight reciprocating means, wherein the adaptor has a coupling groove into which a coupling projection of the straight reciprocating member is press-fitted.

According to a facial bone surgery apparatus for medical treatment of the present invention, it is possible to perform bone cutting in a direction different from an introduction direction of a cutting tool because a bent section is formed at an outer pipe to enable power transmission in a direction different from a moving direction of a straight reciprocating member of a hand piece. In addition, it is possible to perform various surgical operations under the condition of a limited incision or approach angle because an assembly angle of the cutting tool and an assembly angle of the outer pipe can be implemented in various embodiments.

The foregoing and other objects and advantages of the invention and the manner in which the same are accomplished will become clearer based on the following detailed description taken in conjunction with the accompanying drawings.

### BRIEF DESCRIPTION OF THE DRAWINGS

FIG. 1 is a front view of a facial bone surgery apparatus adapted to a hand piece in accordance with the present invention;

FIG. 2 is a cross-sectional view of the facial bone surgery apparatus in accordance with the present invention;

FIG. 3 is an exploded perspective view of a cutting tool of the facial bone surgery apparatus in accordance with the present invention;

FIG. 4 is a front view showing an assembly direction change operation of the cutting tool of the facial bone surgery apparatus in accordance with the present invention;

FIG. 5 is a perspective view of another cutting tool used in the facial bone surgery apparatus in accordance with the present invention;

FIG. 6 is an exploded perspective view of an outer pipe assembly of the facial bone surgery apparatus in accordance with the present invention;

FIGS. 7 and 8 are cross-sectional views showing the outer pipe assembly of the facial bone surgery apparatus in accordance with the present invention;

FIG. 9 is a cross-sectional view showing an outer pipe assembly angle change operation of the facial bone surgery apparatus; and

FIG. 10 is an exploded perspective view of an inner pipe assembly of the facial bone surgery apparatus.

### DETAILED DESCRIPTION

Exemplary embodiments of the present invention will now be described in detail with reference to the accompanying drawings.

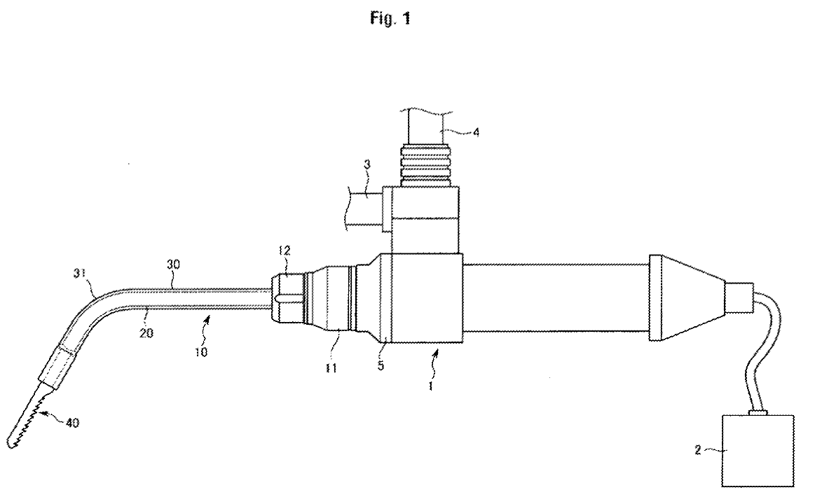

FIG. 1 is a front view of a facial bone surgery apparatus adapted to a hand piece in accordance with the present invention. As shown, the facial bone surgery apparatus 10 in accordance with the present invention is detachably assembled to a hand piece 1 by a connection means including a connection tube 11 and a fixing nut 12.

The hand piece 1 is an example of Korean Patent Registration No. 589118 issued to the inventor. While not shown in FIG. 1, the hand piece 1 includes a straight reciprocating means driven by power of an external drive means 2, a saline solution supply means connected to a saline solution supply connection pipe 3 to supply a saline solution into the surgery apparatus, and a foreign substance suction means connected to a suction unit connection pipe to suck foreign substances generated during surgical operations.

The facial bone surgery apparatus 10 in accordance with the present invention connected to the hand piece 1 is formed in a dual pipe structure comprised of an inner pipe 20 and an outer pipe 30. The inner pipe 20 is an elongated hollow pipe, and a cutting tool 40 for an operation is detachably coupled to one end of the inner pipe 20. The inner pipe 20 is formed of a flexible material and linked to the straight reciprocating means of the hand piece 1 to reciprocate in the outer pipe 30 and to be bent in a bent section.

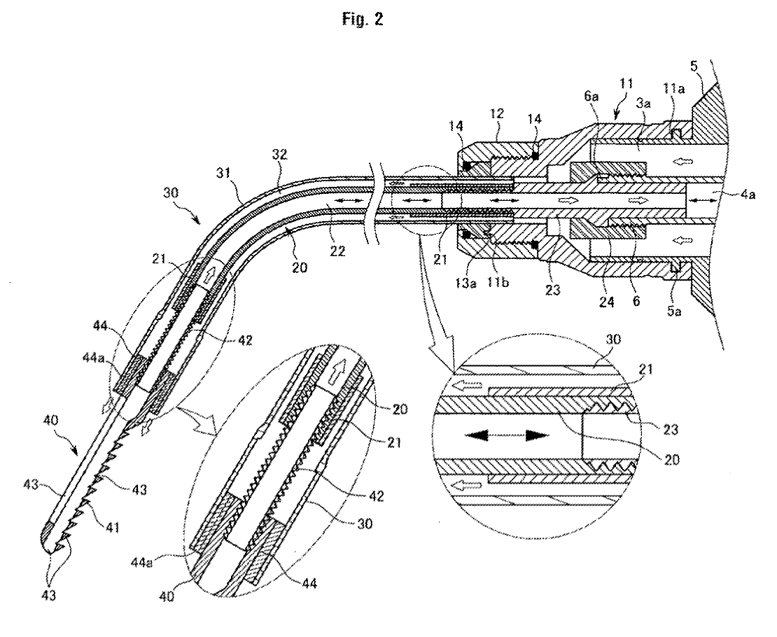

For this purpose, for example, the inner pipe 20 may be formed of a plastic material, such as Teflon or polypropylene. The material has a certain stiffness to be bent at the bent section and to transmit reciprocal movement in an axial direction, even though the elongated pipe is formed of the same material. In addition, as shown in FIG. 2, reinforcement bushings 21 are fixed to both ends of the inner pipe 20 to increase structural intensity of the both ends, thereby securely transmitting or receiving the reciprocal movement in an axial direction.

The outer pipe 30 may be formed of a metal material such as stainless steel, which has high corrosion resistance and low bending deformability. The outer pipe 30 is formed of an elongated hollow pipe, and disposed on an outer periphery of the inner pipe 20 to guide reciprocal movement of the inner pipe 20. The outer pipe 30 has a bent section 31 formed between both ends thereof such that a cutting direction by the cutting tool 40 is different from a direction approaching a surgical operation portion through an incision part. The bent section 31 may be formed adjacent to the cutting tool 40.

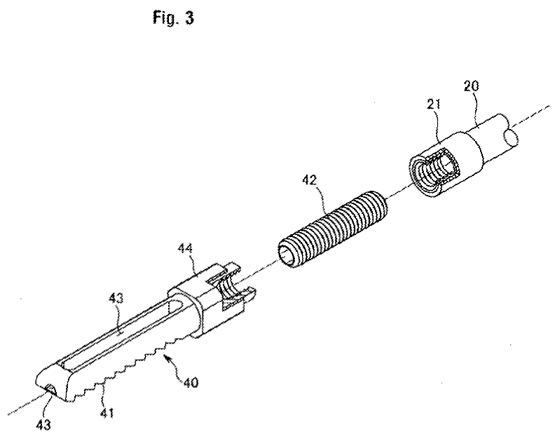

The cutting tool 40 is detachably assembled to an end of the inner pipe 20. As shown in FIGS. 2 and 3, the cutting tool 40 includes a cutting blade 41 for grinding and cutting a bone. One end of the cutting tool 40 is assembled to the inner pipe 20 by a connector 42. The connector 42 is threadedly engaged with the inner pipe 20 at its one end and threadedly engaged with the cutting tool 40 at the other end to detachably assemble the cutting tool 40 to the inner pipe 20, and transmits reciprocal movement of the inner pipe 20 to the cutting tool 40 to perform facial bone surgery using the cutting blade 41.

In addition, the cutting tool 40 and the connector 42 have a hollow structure to be in communication with the interior of the inner pipe 20 used as a foreign substance suction path 22. The cutting tool 40 has a through-hole 43 in communication with the interior and exterior thereof to suck external foreign substances into the interior.

A straight guide member 44 is fixed to an outer surface of the cutting tool 40 and press-fitted into the outer pipe 30 to guide reciprocal movement. A cutting blade direction setting structure is formed between the straight guide member 44 and the outer pipe 30 to set an assembly angle of the cutting tool 40 in a circumferential direction about an assembly axis a of the cutting tool 40.

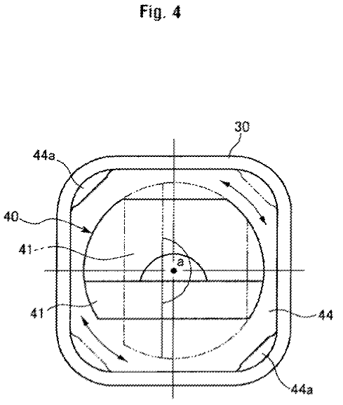

As shown in FIG. 4, in the cutting blade direction setting structure, while the straight guide member 44 and the outer pipe 30 have a rectangular cross-section to set a direction of the cutting blade 41 of the cutting tool 40 at equal 90° intervals in a circumferential direction about the assembly axis a of the outer pipe 30, but not limited thereto, directions of the cutting blades 41 can be variously set through a design change of the cross-sectional shape.

In addition, a predetermined gap is defined between the inner pipe 20 and the outer pipe 30 to form a saline solution supply path 32. As shown in FIG. 4, gaps 44a are formed between the straight guide member 44 and the outer pipe 30 to be in communication with the saline solution supply path 32 by forming grooves in the straight guide member 44. Therefore, the saline solution supplied into the saline solution supply path 32 can be supplied into a surgery portion through the gaps 44a.

Further, a foreign substance suction path 22 is formed in the inner pipe 20, and connected to the interior and the through-hole 43 of the cutting tool 40 to suck and remove foreign substances such as bone fragments and a saline solution of the a surgical operation portion.

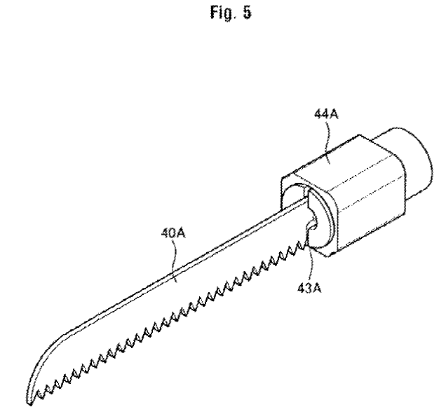

Since the cutting tool 40 is detachably assembled to the inner pipe 20 by the connector 42, as shown in FIG. 5, another cutting tool 40A may be detachably assembled to the inner pipe 20 by the connector 42. A through-hole 43A in communication with the foreign substance suction path 22 of the inner pipe 20 may be appropriately formed depending on the shape of the cutting tool. For example, the through-hole 43A is formed between the cutting tool 40A and the straight guide member 44A.

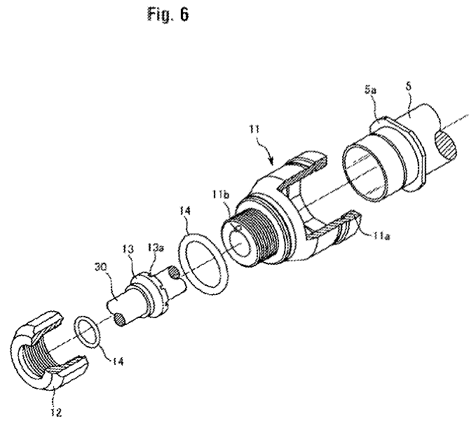

Meanwhile, as shown in FIGS. 2 and 6, an assembly structure of the outer pipe 30 to the main body 5 of the hand piece 1 includes a fixing support 13 fixed to an outer periphery of the outer pipe 30, a connection tube 11 detachably coupled to the main body 5 of the hand piece 1 at one side and having a threaded part formed at the other side, and a fixing nut 12 hooked by the fixing support 13 and threadedly engaged with the threaded part of the connection tube 11 to couple the outer pipe 30 to the connection tube 11.

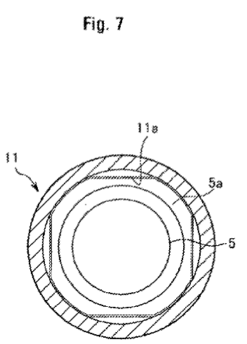

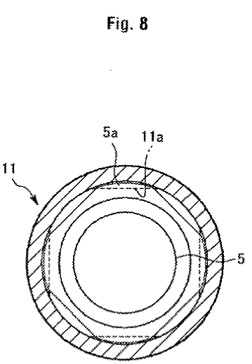

At this time, as shown in FIGS. 7 and 8, in a coupling and separation structure of the connection tube 11 and the main body 5, hooking grooves 11a corresponding to four projections 5a formed at an outer periphery of the main body 5 at 45° intervals are formed at an inner periphery of the connection tube 11, the connection tube 11 is rotated 45° clockwise and counter clockwise, with the projections 5a of the main body 5 being inserted into the hooking grooves 11a of the connection tube 11, such that the projections 5a are hooked by or released from the hooking grooves 11a to be coupled thereto or separated therefrom.

As described above, the assembly of the outer pipe 30 of the surgery apparatus 10 to the main body 5 of the hand piece 1 enables communication of the saline solution supply path 3a of the saline solution supply means connected to the saline solution supply connection pipe 3 with the saline solution supply path 32 of the surgery apparatus 10, thereby supplying a saline solution into a surgical operation portion. At this time, a packing 14 may be installed between the fixing nut 12, the outer pipe 30, and the connection tube 11 to maintain a water tight seal therebetween.

In addition, a bending direction setting structure is disposed between the fixing support 13 and the connection tube 11 to vary an assembly angle of the outer pipe 30 in a circumferential direction about an assembly axis b of the outer pipe 30.

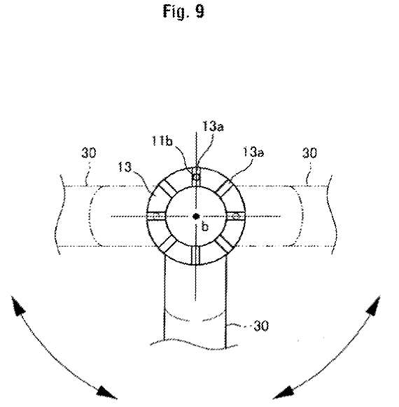

As shown in FIGS. 6 and 9, the bending direction setting structure is comprised of a plurality of insertion grooves 13a radially formed on a coupling surface of the fixing support 13 about the assembly axis b, and an insertion projection lib projecting from the coupling surface of the connection tube 11 opposite to the insertion grooves 13a to be inserted into one of the insertion grooves 13a. Therefore, the insertion projection lib is selectively inserted into one of the insertion grooves 13a to vary a bending direction of the outer pipe 30 with respect to the main body 5 of the hand piece 1.

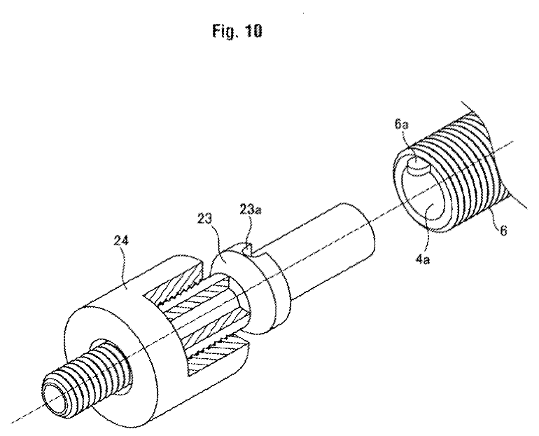

As shown in FIGS. 2 and 10, a connection and separation structure of the inner pipe 20 and the hand piece 1 includes a hollow adaptor 23 for connecting the straight reciprocating member 6 of the straight reciprocating means installed at the hand piece 1 to the inner pipe 20, and a locking nut 24 threadedly engaged with the straight reciprocating member 6 to fix the adaptor 23 to the straight reciprocating member 6. In addition, the adaptor 23 has a coupling groove 23a coupled to a coupling projection 6a of the straight reciprocating member 6.

By means of the connection structure of the inner pipe 20, the foreign substance suction path 4a of the foreign substance suction means connected to the suction unit connection pipe 4 is in communication with the foreign substance suction path 22 of the surgery apparatus 10 to suck and remove foreign substances such as the bone fragments and the saline solution in the surgical operation portion.

Using the facial bone surgery apparatus 10 in accordance with the present invention, as shown in FIGS. 1 and 2, when the drive means 2 reciprocates the straight reciprocating member 6 of the straight reciprocating means installed at the hand piece 1, the inner pipe 20 connected thereto by the adaptor 23 also reciprocates. As a result, the cutting tool 40 assembled to an end of the inner pipe 20 reciprocates to perform a grinding or cutting operation of a facial bone using the cutting blade 41.

In addition, at the same time the surgical operation is performed, a saline solution is supplied into the surgical operation portion through the saline solution supply path 32 and the gap 44a of the surgery apparatus 10 in communication with the saline supply path 3a of the hand piece 1, and foreign substances such as the bone fragments and saline solution generated during the surgical operation are discharged through the foreign substance suction path 22 of the surgery apparatus 10 in communication with the foreign substance suction path 4a of the hand piece 4a.

At this time, since the outer pipe 30 has the bent section 31 and the flexible inner pipe 20 transmits reciprocal movement of the hand piece 1 to another cutting tool 40 having a movement direction different from the reciprocal movement, a surgical operation having a cutting direction of the facial bone different from an introduction direction of the cutting tool 40 through the incision part can be performed.

In addition, as shown in FIGS. 3 and 4, since the assembly angle of the cutting tool 40 can be varied in a circumferential direction about the assembly axis a, it is possible to freely set a direction of the cutting blade 41 of the cutting tool 40 with respect to the surgical operation portion regardless of an introduction direction of the outer pipe 30.

Further, as shown in FIGS. 6 and 9, since the assembly angle of the outer pipe 30 can be varied in a circumferential direction about the assembly axis b, it is possible to vary the assembly angle into a direction convenient to enter the bent direction of the outer pipe 30 depending on the incision part and the surgical operation portion.

As described above, since the outer pipe 30 is bent to make the introduction direction of the cutting tool 40 different from the cutting direction, and the direction of the cutting blade 41 of the cutting tool 40 and the bending direction of the outer pipe 30 can be changed in a variety of different ways, it is possible to perform a facial bone surgery under the condition of a limited incision or approach angle. In addition, since the cutting tool 40 can be detachably attached to the inner pipe 20, another type of cutting tool can be simply and readily exchanged.

Further, if a design of the connection structure of the outer pipe 30 and the inner pipe 20 in addition to the hand piece 1 is changed, the cutting tool can be connected to another type of hand piece 1. While this invention has been described with reference to exemplary embodiments thereof, it will be clear to those of ordinary skill in the art to which the invention pertains that various modifications may be made to the described embodiments without departing from the spirit and scope of the invention as defined in the appended claims and their equivalents.

As can be seen from the foregoing, a bent section is formed at an insertion part to be inserted into an incision part, and power transmission can be performed in the bent section such that a cutting operation having a different angle than a moving direction of a straight reciprocating member installed at an hand piece can be performed, thereby performing a surgical operation under the condition of a limited incision or approach angle.

1. A facial bone surgery apparatus for medical treatment assembled to a hand piece including a straight reciprocating means, a saline solution supply means and a foreign substance discharge means, the facial bone surgery apparatus comprising:
an inner pipe reciprocally movable together with the straight reciprocating means and having a hollow shape of a soft flexible material;
an outer pipe connected to a main body of the hand piece and installed at an outer periphery of the inner pipe to guide reciprocal movement of the inner pipe, and having a bent section formed between both ends thereof, the outer pipe being formed of a hard material; and
a cutting tool assembled at an end of the inner pipe to reciprocate therewith and reciprocally guided by the outer pipe.

2. The facial bone surgery apparatus for medical treatment according to claim 1, further comprising a straight guide member fixed to an outer surface of the cutting tool to be press-fitted into the outer pipe, wherein a cutting blade direction setting structure is formed between the straight guide member and the outer pipe to vary the direction of a cutting blade of the cutting tool in a circumferential direction about an assembly axis of the cutting tool.

3. The facial bone surgery apparatus for medical treatment according to claim 2, wherein the cutting blade direction setting structure and the straight guide member have a polygonal outer cross-section.

4. The facial bone surgery apparatus for medical treatment according to claim 1, wherein reinforcement bushings are formed at outer peripheries of both ends of the inner pipe.

5. The facial bone surgery apparatus for medical treatment according to claim 2, wherein a saline solution supply path is formed in a gap between the outer pipe and the inner pipe, and is in communication with a gap formed between the outer pipe and the straight guide member to supply a saline solution.

6. The facial bone surgery apparatus for medical treatment according to claim 2, wherein a foreign substance suction path is formed in the inner pipe, and a through-hole is formed at the cutting tool to be in communication with the foreign substance suction path of the inner pipe.

7. The facial bone surgery apparatus for medical treatment according to claim 1 further comprising a connection structure connecting the outer pipe and the hand piece, the connection structure comprising:
a fixing support fixed to an outer periphery of the outer pipe;
a connection tube detachably coupled to the main body of the hand piece at one side and having a threaded part formed at the other side;
a fixing nut hooked by the fixing support and threadedly engaged with the threaded part of the connection tube to couple the outer pipe to the connection tube; and
a packing for maintaining a water tight seal between the fixing nut, the outer pipe and the connection tube.

8. The facial bone surgery apparatus for medical treatment according to claim 7, wherein a bending direction setting structure is formed between the fixing support and the connection tube to vary an assembly angle of the outer pipe in a circumferential direction about an assembly axis of the outer pipe.

9. The facial bone surgery apparatus for medical treatment according to claim 8, wherein the bending direction setting structure comprises:
a plurality of insertion grooves formed at the fixing support of the outer pipe in a circumferential direction about an assembly axis thereof; and
an insertion projection formed at the connection tube to be press-fitted into the insertion grooves.

10. The facial bone surgery apparatus for medical treatment according to claim 1 further comprising a connection structure connecting the inner pipe and the hand piece, the connection structure comprising:
an adaptor for connecting a straight reciprocating member of the straight reciprocating means installed at the hand piece to the inner pipe, the adapter providing communication between a foreign substance suction path formed in the hand piece with a foreign substance suction path formed in the inner pipe; and
a locking nut for threadedly engaging the adaptor with the straight reciprocating means, wherein the adaptor has a coupling groove into which a coupling projection of the straight reciprocating member is press-fitted.
